(19) United States
(12) Patent Application Publication
(10) Pub. No.: US 2026/0219258 A1
(43) Pub. Date: Jul. 30, 2026

(54) BIOLOGICAL INFORMATION MEASUREMENT SYSTEM AND TOILET SEAT DEVICE

(71) Applicant: TOTO LTD., Kitakyushu-shi, Fukuoka (JP)

(72) Inventors: Daiki Aoki, Fukuoka (JP); Haruna Kawasaki, Fukuoka (JP); Koki Nagano, Fukuoka (JP); Shota Fujino, Fukuoka (JP); Hitoaki Higuchi, Fukuoka (JP); Satoko Kizuka, Fukuoka (JP); Masamichi Tosaki, Fukuoka (JP)

(21) Appl. No.: 19/149,109

(22) PCT Filed: Jan. 19, 2024
(86) PCT No.: PCT/JP2024/001493

§ 371 Date: Jul. 18, 2025

(30) Foreign Application Priority Data
Mar. 24, 2023 (JP) 2023-047625

(51) Int. Cl. G01N 33/497; E03D 9/00

(52) U.S. Cl. CPC G01N 33/4975; E03D 9/00

(57) ABSTRACT

A biological information measurement system according to an embodiment is a biological information measurement system that measures biological information of a user in a toilet room based on an intestinal gas emitted into a bowl of a closet bowl installed in the toilet room, the biological information measurement system including: a gas detection device that includes a gas sensor that reacts with a gas contained in a gas mixture; and a control device that controls the gas detection device, in which the gas sensor includes a sensor element and a resistor element for measurement, and the control device performs reference value control for controlling the gas detection device such that a measurement value of the gas sensor becomes a value within a predetermined range while the user does not use the closet bowl and controlling the measurement value used as a reference value to be a predetermined value.

### FIELD

The disclosed embodiment relates to a biological information measurement system and a toilet seat device.

### BACKGROUND

Hitherto, there is known a biological information measurement system that senses fecal gas emitted when a user of a toilet defecates by using a gas sensor and measures a physical condition of the user (see, for example, Patent Literature 1). Further, a gas detection system that sets a resistance value of a resistor element and a resistance value of a sensor element so as to be equal to each other in a gas sensor is known (see, for example, Patent Literature 2).

### CITATION LIST

### Patent Literature

Patent Literature 1: JP 6674623 B2
Patent Literature 2: JP 2022-174045 A

### SUMMARY

### Technical Problem

However, there is room for improvement in the above-described technologies according to the related art. For example, simply setting the resistance value of the resistor element and the resistance value of the sensor element so as to be equal to each other may make it challenging to accommodate a change in an environment for measuring the fecal gas, and a measurement value of the gas sensor may fluctuate depending on the change in the environment or the like. In such a case, a resolution of the gas sensor is not stable, and it is difficult to perform appropriate measurement, for example, in a case where it is desired to observe a temporal change, and thus, there is room for improvement. Therefore, it is desired to appropriately execute processing related to the gas measurement.

An object of the disclosed embodiment is to provide a biological information measurement system and a toilet seat device capable of appropriately executing processing related to gas measurement.

### Solution to Problem

A biological information measurement system according to one aspect of an embodiment that measures biological information of a user in a toilet room based on a fecal gas emitted into a bowl of a closet bowl installed in the toilet room, the biological information measurement system includes a gas detection device that includes a gas sensor that reacts with a gas contained in a gas mixture; and a control device that controls the gas detection device, wherein the gas sensor includes a sensor element and a resistor element for measurement, the control device performs reference value control for controlling the gas detection device such that a measurement value of the gas sensor becomes a value within a predetermined range while the user does not use the closet bowl and controlling the measurement value used as a reference value to be a predetermined value, and the gas detection device executes processing related to fecal gas measurement by using the reference value controlled by the reference value control.

With the biological information measurement system according to one aspect of the embodiment, even in a case where a detection value for the gas sensor at the time of non-defecation fluctuates due to a temperature and humidity condition of a toilet space or an air freshener, the reference value of the gas sensor is controlled within a predetermined range at a predetermined timing, whereby the resolution change for each measurement can be suppressed. Therefore, with the biological information measurement system according to one aspect of the embodiment, even if environmental conditions of the toilet space are different for each measurement at the time of defecation, it is possible to accurately detect the fecal gas, to standardize daily measurement conditions for the fecal gas, and to accurately capture a temporal change in a physical condition of the user obtained from the fecal gas. Therefore, the biological information measurement system can appropriately execute processing related to the gas measurement.

The inventors have continued studies as to whether or not it is possible to accurately measure the physical condition of a toilet user from the fecal gas as described in Patent Literature 1 and the like. In the study, it has been found that when the fecal gas is detected in a toilet as in Patent Literature 1, since the gas mixture in the bowl of the closet bowl is sucked and the gas is detected, the gas sensor is affected not only by the fecal gas to be detected but also by toilet-specific noise such as the temperature and humidity condition in the toilet space, a residual gas, the air freshener, a human-derived perfume, and an air disturbance caused by a motion of the user. As a result, the detection value serving as the reference for the gas sensor fluctuates, leading to a significant resolution change, and accuracy in measurement of a health condition of the user based on the fecal gas significantly decreases when confirming a temporal change. On the other hand, by adopting the configuration of Patent Literature 2, the resistance values of the resistor element and the sensor element can be made equal to each other, and the resolution of the detection value for the fecal gas per measurement can be improved. However, the reference value changes every time the measurement is performed, which leads to the resolution change, and thus, there is a problem that it is not possible to evaluate a temporal change of fecal gas information. Therefore, with the biological information measurement system according to one aspect of the embodiment, it is possible to accurately measure the temporal change of the amount of the fecal gas even when affected by the noise such as an odor gas and the air freshener remaining in the toilet space.

In the biological information measurement system according to one aspect of the embodiment, the control device performs the reference value control for controlling the reference value to be the predetermined value by changing a resistance value of the resistor element of the gas sensor.

With the biological information measurement system according to one aspect of the embodiment, the reference value control for controlling the reference value to be the predetermined value by changing the resistance value of the resistor element of the gas sensor can be appropriately performed. Therefore, the biological information measurement system can appropriately execute processing related to the gas measurement.

In the biological information measurement system according to one aspect of the embodiment, the control device performs the reference value control every time the fecal gas measurement ends.

With the biological information measurement system according to one aspect of the embodiment, the reference value control can be performed for each defecation act, and the measurement can be performed while suppressing the resolution change for each fecal gas detection. For example, it is possible to cope with a toilet environment that changes each time such as an influence of odor of the previous user (perfume, the fecal gas, or the like of the previous user), and more accurately capture the temporal change in the physical condition of the user obtained from the fecal gas. Therefore, the biological information measurement system can appropriately execute processing related to the gas measurement.

In the biological information measurement system according to one aspect of the embodiment, the control device performs the reference value control by executing processing of feeding back the measurement value of the gas sensor.

With the biological information measurement system according to one aspect of the embodiment, since the reference value can be controlled based on the detection value of the gas sensor currently output by the feedback control, the reference value can be adjusted with higher accuracy. Therefore, the reference value control can be performed with high accuracy based on the detection value of the gas sensor, and the measurement can be performed while suppressing the resolution change at the time of detecting the fecal gas. Therefore, the biological information measurement system can appropriately execute processing related to the gas measurement.

In the biological information measurement system according to one aspect of the embodiment, the control device acquires defecation act use prediction information related to information for predicting a defecation act of the user, and performs the reference value control when the defecation act use prediction information is acquired.

With the biological information measurement system according to one aspect of the embodiment, it is possible to perform sensing before the defecation act and perform the reference value control, and it is possible to perform the measurement while effectively suppressing the resolution change at the time of detecting the fecal gas. Therefore, the biological information measurement system can appropriately execute processing related to the gas measurement.

The biological information measurement system according to one aspect of the embodiment, includes seating sensing means that senses seating of the user on a toilet seat of the closet bowl, wherein the control device acquires the defecation act use prediction information based on the sensing performed by the seating sensing means, and performs the reference value control when the defecation act use prediction information is acquired.

With the biological information measurement system according to one aspect of the embodiment, it is possible to perform the reference value control at the timing of sensing at which a change in the air is small before the defecation act, and it is possible to perform the measurement while more effectively suppressing the resolution change at the time of the detecting the fecal gas. Therefore, the biological information measurement system can appropriately execute processing related to the gas measurement.

A toilet seat device according to one aspect of an embodiment that measures biological information of a user in a toilet room based on a fecal gas emitted into a bowl of a closet bowl installed in the toilet room, the toilet seat device includes a gas detection device that includes a gas sensor that reacts with a gas contained in a gas mixture; and a control device that controls the gas detection device, wherein the gas sensor includes a sensor element and a resistor element for measurement, the control device performs reference value control for controlling the gas detection device such that a measurement value of the gas sensor becomes a value within a predetermined range while the user does not use the closet bowl and controlling the measurement value used as a reference value to be a predetermined value, and the gas detection device executes processing related to fecal gas measurement by using the reference value controlled by the reference value control.

With the toilet seat device according to one aspect of the embodiment, even in a case where the detection value for the gas sensor at the time of non-defecation fluctuates due to an influence of the temperature and humidity condition of the toilet space or the air freshener, the reference value of the gas sensor is controlled within a predetermined range at a predetermined timing, whereby the resolution change for each measurement can be suppressed. Therefore, with the toilet seat device according to one aspect of the embodiment, even if the environmental conditions of the toilet space are different for each measurement at the time of defecation, it is possible to accurately detect the fecal gas, to standardize the daily measurement conditions for the fecal gas, and to accurately capture the temporal change in the physical condition of the user obtained from the fecal gas.

Therefore, the toilet seat device can appropriately execute processing related to the gas measurement.

### Advantageous Effects of Invention

According to one aspect of the embodiment, processing related to gas measurement can be appropriately executed.

### BRIEF DESCRIPTION OF DRAWINGS

FIG. 1 is a perspective view illustrating an example of a configuration of a toilet room according to an embodiment.

FIG. 2 is a plan view illustrating an example of a configuration of a measurement device according to the embodiment.

FIG. 3 is a diagram illustrating an example of an overall outline of a biological information measurement system according to the embodiment.

FIG. 4 is a diagram illustrating an example of a relationship between an action of a user and an operation of the system.

FIG. 5 is a block diagram illustrating an example of a configuration of a toilet seat device according to the embodiment.

FIG. 6 is a block diagram illustrating an example of a configuration of a control device according to the embodiment.

FIG. 7 is a diagram illustrating an example of a configuration of a gas sensor.

FIG. 8 is a diagram illustrating an example of reference value control according to the embodiment.

FIG. 9 is a diagram illustrating an example of a timing of the reference value control according to the embodiment.

FIG. 10 is a diagram illustrating feedback control of a reference value.

FIG. 11 is a diagram illustrating an example of an action before a defecation act.

### DESCRIPTION OF EMBODIMENTS

Hereinafter, embodiments of a biological information measurement system and a toilet seat device disclosed in the present application will be described in detail with reference to the accompanying drawings. Note that the present invention is not limited by the following embodiments. In the present application, a gas that is derived from intestinal fermentation and indicates a high level of health is referred to as a health-related gas, and a gas that is derived from intestinal putrefaction and indicates a low level of health is referred to as an odoriferous gas.

For example, the health-related gas is a gas generated by fermentation by good bacteria in the intestine. For example, the health-related gas may be a gas that is derived from the intestinal fermentation and increases as the level of health in the intestine increases. Specific examples of the health-related gas include hydrogen, carbon dioxide, acetic acid, methane, ethanol, and water.

In addition, for example, the odoriferous gas is a gas generated by fermentation by bad bacteria in the intestine. For example, the odoriferous gas may be a gas containing a sulfur component among fecal gases. Examples of the odoriferous gas include ammonia, trimethylamine, hydrogen sulfide, methyl mercaptan, indole, and skatole. The fecal gas described here is a gas that comes out from the intestine, and for example, the fecal gas includes a gas that comes out during defecation and a gas that is not emitted during the defecation.

### 1. Embodiment

Hereinafter, an outline of a toilet room R as a gas collection place and a biological information measurement system 1 will be described, and then various steps of processing executed by the biological information measurement system 1 and a configuration for executing the steps of processing will be described.

### 1-1. Configuration Example of Toilet Room

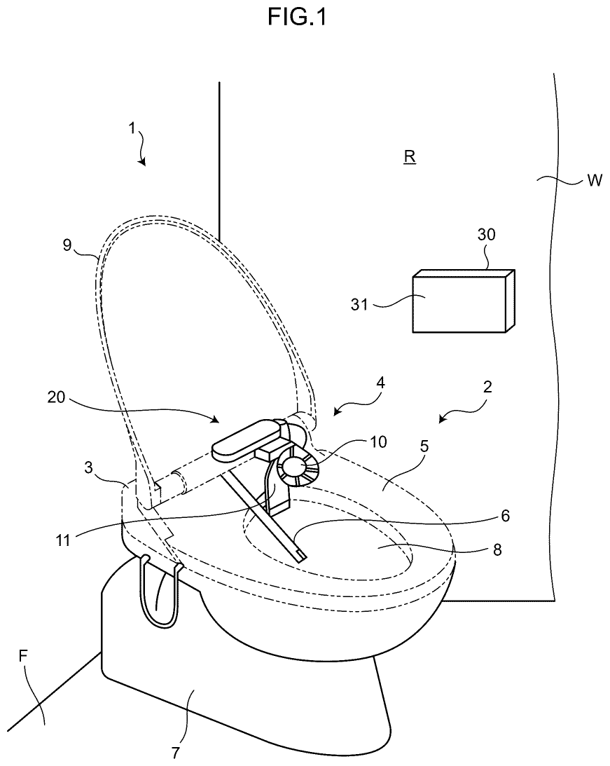

First, a configuration of a biological information measurement system according to an embodiment will be described with reference to FIG. 1. FIG. 1 is a perspective view illustrating an example of a configuration of the biological information measurement system according to the embodiment; In FIG. 1, a measurement device 4 is illustrated in a see-through representation through a toilet seat 5 and a toilet lid 9 in order to illustrate a configuration of the measurement device 4.

As illustrated in FIG. 1, a toilet bowl 7 is installed on a floor surface F in the toilet room R. Hereinafter, a direction facing a space of the toilet room R from the floor surface F may be referred to as an upper side. Components of the biological information measurement system 1 such as the measurement device 4 that performs gas detection and includes a suction device 10 and a gas detection device 20 are disposed in the toilet room R.

The toilet bowl 7 is a closet bowl, and a bowl portion 8 is formed in the toilet bowl 7. The bowl portion 8 has a shape recessed downward and is a portion that receives excreta of a user. The toilet bowl 7 is not limited to a floor-mounted type as illustrated, and may be of any type as long as the biological information measurement system 1 can be applied, such as a wall-mounted type. The toilet bowl 7 is provided with a rim portion over an entire circumference of an end portion of an opening that the bowl portion 8 faces. In the toilet room R, for example, a cleaning water tank for storing cleaning water may be installed in the vicinity of the toilet bowl 7, or a so-called tank-less type in which no cleaning water tank is installed may be used.

For example, when a cleaning operation unit (not illustrated) for cleaning provided in the toilet room R is operated by the user, cleaning water is supplied to the bowl portion 8 of the toilet bowl 7, thereby performing toilet bowl cleaning. The cleaning operation unit may be an operation lever, or a touch operation may be made on a toilet bowl cleaning object displayed on an operation device 30. The cleaning operation unit is not limited to one that is manually operated by the user to perform the toilet bowl cleaning like the operation lever, and may be one that senses a human body using a sensor that senses the user like a seating sensor to perform the toilet bowl cleaning.

A toilet seat device 2 is attached to an upper portion of the toilet bowl 7, and includes a main body portion 3, the measurement device 4, the toilet seat 5, and a cleaning nozzle 6. The toilet seat device 2 is placed on the upper portion of the toilet bowl 7 in which the bowl portion 8 for receiving the excreta is formed. The toilet seat device 2 is placed on the upper portion of the toilet bowl 7 so as to be extended to the bowl portion 8 before the cleaning nozzle 6 sprays cleaning water. The toilet seat device 2 may be detachably attached to the toilet bowl 7 or may be integrally attached to the toilet bowl 7.

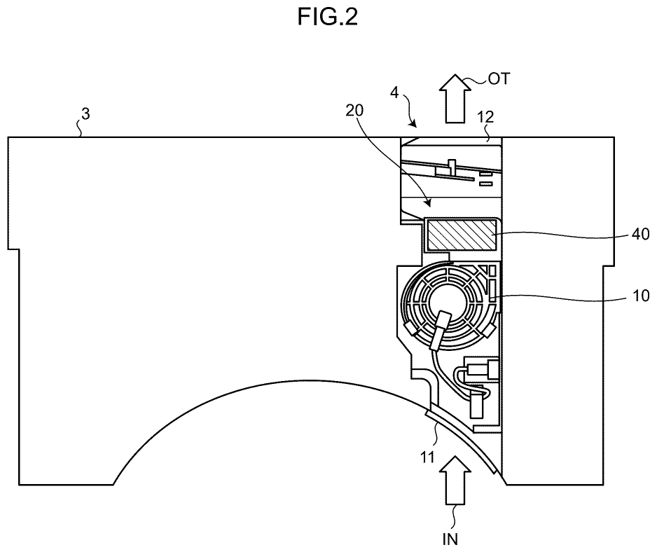

The toilet seat device 2 measures biological information of the user in the toilet room R based on the fecal gas emitted into the bowl portion 8 of the toilet bowl 7 installed in the toilet room R by the configuration of the measurement device 4 and the like. The measurement device 4 includes the suction device 10 and the gas detection device 20. The measurement device 4 will be described in detail with reference to FIG. 2.

As illustrated in FIG. 1, the toilet seat 5 is formed in an annular shape, and is disposed at a position overlapping the opening of the toilet bowl 7 along an end portion (rim portion) of the bowl portion 8. The user is seated on the toilet seat 5. The toilet seat 5 functions as a seating portion that supports the buttocks of the seated user. The toilet lid 9 is attached to the toilet seat device 2 as necessary, and the toilet seat device 2 does not have to include the toilet lid 9.

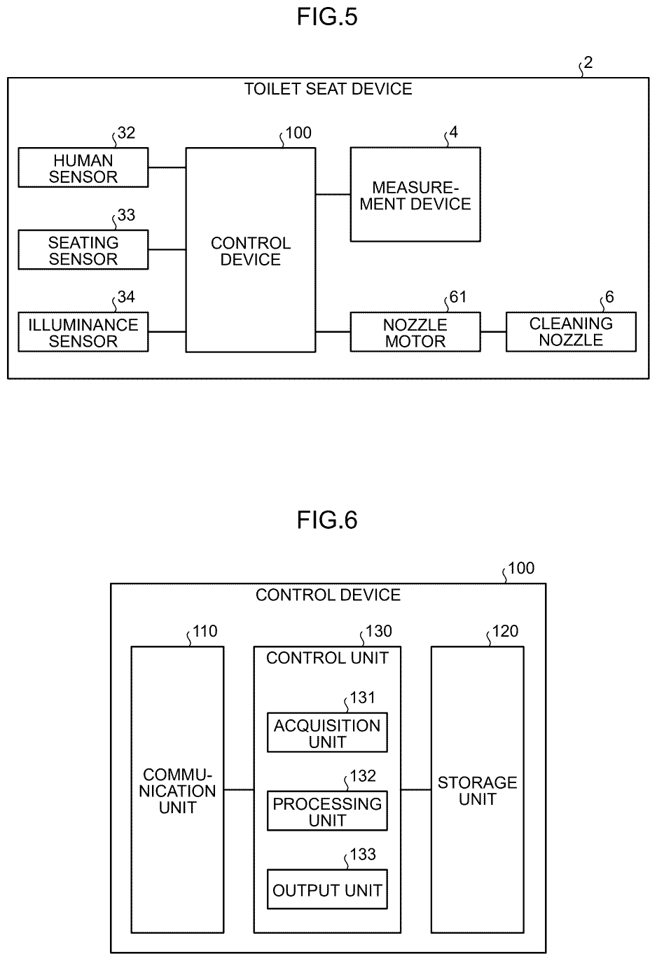

The cleaning nozzle 6 is a nozzle for ejecting water for cleaning. The cleaning nozzle 6 is configured to be movable forward and backward with respect to a housing of the main body portion 3 by driving of a drive source (such as a nozzle motor 61 in FIG. 5) such as an electric motor. The cleaning nozzle 6 is connected to a water source such as a water pipe (not illustrated). As illustrated in FIG. 1, when the cleaning nozzle 6 is at a position (also referred to as “extended position”) at which the cleaning nozzle 6 is extended with respect to the housing of the main body portion 3, the cleaning nozzle 6 sprays the water from the water source to the body of the user to clean the private part.

FIG. 1 illustrates a state in which the cleaning nozzle 6 is at the extended position. The cleaning nozzle 6 may also be used for cleaning the inside of the toilet bowl 7 (the bowl portion 8 or the like). The cleaning nozzle 6 may be used so as to be able to switch between a private part cleaning mode for cleaning the private part of the user and a toilet bowl cleaning mode for spraying the water into the toilet bowl 7. For example, the cleaning nozzle 6 may be used so as to be able to switch between the private part cleaning mode and the toilet bowl cleaning mode under the control of the toilet seat device 2.

The operation device 30 is provided in the toilet room R. The operation device 30 is provided at a position where the user can operate the operation device 30. The operation device 30 is provided at a position where the user can operate the operation device 30 when being seated on the toilet seat 5. In FIG. 1, the operation device 30 is disposed on a wall surface W on a left side when viewed from the user seated on the toilet seat 5. The operation device 30 is not limited to being disposed on the wall surface and may be disposed in various modes as long as the user seated on the toilet seat 5 can use the operation device 30. For example, the operation device 30 may be provided integrally with the toilet seat device 2.

The operation device 30 is communicably connected to the toilet seat device 2 via a predetermined network in a wired or wireless manner. For example, the toilet seat device 2 and the operation device 30 may be connected to each other in any manner as long as information can be transmitted and received, and may be connected to each other in a wired or wireless manner.

The operation device 30 receives various operations from the user via a display surface (for example, a display screen 31) using a touch panel function, for example. Furthermore, the operation device 30 may include a switch or a button, and may receive various operations through the switch, the button, or the like. The display screen 31 is a display screen of a tablet terminal or the like implemented by, for example, a liquid crystal display or an organic electro-luminescence (EL) display, and is a display device for displaying various types of information. That is, the operation device 30 receives an input of the user through the display screen 31 and also performs output to the user. The display screen 31 is a display device that displays various types of information.

The operation device 30 receives a user operation for controlling various functions provided in the toilet room R. The operation device 30 receives a user operation for controlling execution of private part cleaning by the toilet seat device 2. For example, the operation device 30 may include a switch, a button, or the like that receives the user operation described above, and executes various steps of processing according to a user contact with the switch, the button, or the like. The above is an example, and the operation device 30 may receive an operation by the user for executing various steps of processing.

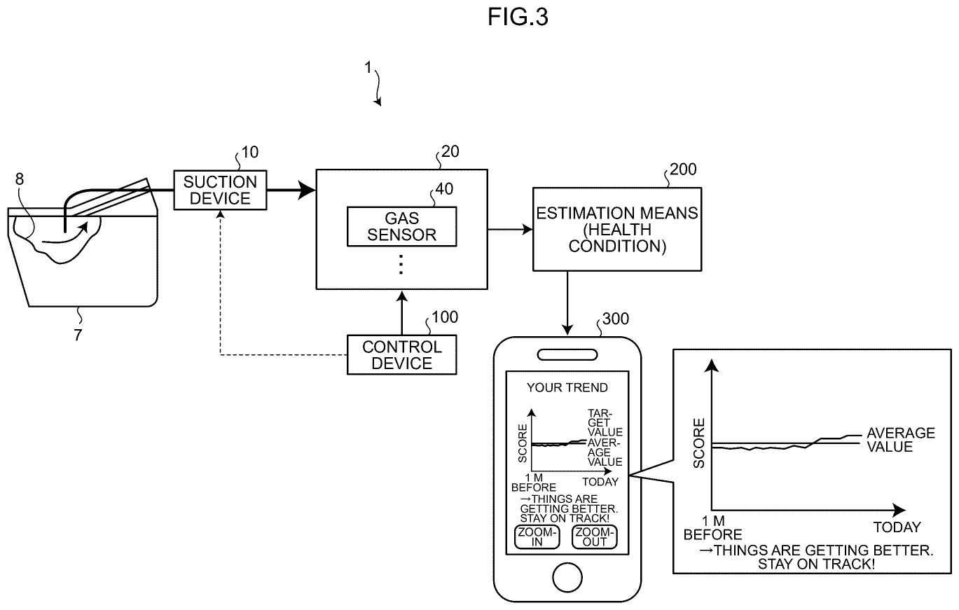

The biological information measurement system 1 measures the biological information of the user in the toilet room R based on the fecal gas emitted into the bowl portion 8 of the toilet bowl 7 installed in the toilet room R by various configurations and processing described below. The biological information measurement system 1 performs control to appropriately measure the fecal gas. The biological information measurement system 1 may provide information to a user terminal (corresponding to display means 300 in FIG. 3) such as a smartphone of the user based on information collected by measurement or the like. In addition, the biological information measurement system 1 may provide information to the operation device 30 (or the display screen 31) of the toilet room R based on the information collected by the measurement or the like.

### 1-2. Configuration of Measurement Device

Next, a configuration of the measurement device 4 will be described with reference to FIG. 2. FIG. 2 is a plan view illustrating an example of the configuration of the measurement device according to the embodiment. In the example illustrated in FIG. 2, a case where the measurement device 4 is disposed in the main body portion 3 is illustrated as an example. In FIG. 2, the configuration of the measurement device 4 is illustrated by removing a portion of the housing (cover) of the main body portion 3 at a position where the measurement device 4 is disposed.

The measurement device 4 includes the suction device 10 that sucks a gas in the bowl portion 8 of the toilet bowl 7 and the gas detection device 20 that detects a component of the sucked gas.

The suction device 10 includes a fan for sucking the gas in the bowl portion 8 of the toilet bowl 7. The suction device 10 is connected to a duct 11 communicating with the inside of the bowl portion 8 of the toilet bowl 7. The duct 11 functions as a flow path for allowing the gas in the bowl portion 8 to flow into the measurement device 4. The suction device 10 drives the fan to suck the gas in the bowl portion 8 by using the duct 11 as the flow path. For example, the suction device 10 executes processing related to the suction under the control of a control device 100. In a case where the suction device 10 doubles as a deodorizing device or the like incorporated in the toilet seat device 2, the suction device 10 may be controlled by control means (device) different from the control device 100.

The gas detection device 20 executes processing related to detection of the component of the gas sucked by the suction device 10. In FIG. 2, the gas detection device 20 is disposed behind the suction device 10 when viewed from the bowl portion 8. FIG. 2 is merely an example, and the gas detection device 20 may be disposed at any position as long as the gas sucked by the suction device 10 can be introduced. The gas detection device 20 is connected to a duct 12 communicating with the outside of the main body portion 3. The duct 12 functions as a flow path for allowing the gas in the gas detection device 20 to flow out from the measurement device 4. For example, the gas in the gas detection device 20 is discharged to the outside of the measurement device 4 using the duct 12 as the flow path in response to driving of the suction device 10.

For example, the gas detection device 20 executes processing related to the gas detection under the control of the control device 100. The gas detection device 20 includes a gas sensor 40 that reacts with a gas contained in a gas mixture. The gas sensor 40 detects a specific component of the gas.

For example, a semiconductor gas sensor is used as the gas sensor 40. The gas sensor 40 may be a hydrogen gas sensor capable of detecting hydrogen. The gas sensor 40 may be an odoriferous gas sensor capable of detecting the odoriferous gas. The gas sensor 40 may be a methane gas sensor capable of detecting methane. For example, the gas detection device 20 includes a plurality of gas sensors 40. The plurality of gas sensors 40 may include a gas sensor 40a that is the hydrogen gas sensor, a gas sensor 40b that is the odoriferous gas sensor, and a gas sensor 40c that is the methane gas sensor. The gas sensors 40a to 40c will be described as the gas sensors 40 unless otherwise specified.

The above is merely an example, and the gas sensor is not limited to the semiconductor gas sensor 40, and a sensor of any aspect may be used. For example, the gas detection device 20 may include a gas sensor such as an infrared CO₂ sensor (carbon dioxide concentration measurement device).

### 1-3. Overall Outline Example of Biological Information Measurement System

Next, an example of an overall outline of the biological information measurement system 1 will be described with reference to FIG. 3. FIG. 3 is a diagram illustrating an example of the overall outline of the biological information measurement system according to the embodiment. A description of points similar to the contents described in FIGS. 1 and 2 will be omitted as appropriate.

In FIG. 3, the biological information measurement system 1 includes the suction device 10, the gas detection device 20, the control device 100, and estimation means 200. FIGS. 1 and 2 illustrate a case where the toilet seat device 2 includes the suction device 10, the gas detection device 20, and the control device 100, but the present disclosure is not limited thereto. For example, the control device 100 may be provided separately from the suction device 10 and the gas detection device 20, and may control the suction device 10 and the gas detection device 20 by communicating with the suction device 10 and the gas detection device 20 in a wireless or wired manner. As described above, the suction device 10 may be controlled by a control unit different from the control device 100.

The estimation means 200 is a computer (information processing device) having a function of executing estimation processing based on information acquired by detection by the gas detection device 20. For example, the estimation means 200 may be a cloud server (server device) positioned outside the toilet room R. In this case, the estimation means 200 is communicably connected to a device (also referred to as “in-toilet device”) disposed in the toilet room R, such as the toilet seat device 2 or the gas detection device 20, via a predetermined network such as the Internet in a wired or wireless manner.

In addition, the estimation means 200 is communicably connected to a device that displays information to the user, such as the display means 300, via a predetermined network in a wired or wireless manner such as the Internet. The estimation means 200 may be communicably connected to the devices such as the in-toilet device and the display means 300 in any manner as long as information can be transmitted and received. The estimation means 200 may be communicably connected in a wired or wireless manner. The estimation means 200 may be communicable with the control device 100.

The estimation means 200 executes the estimation processing regarding a health condition of the user by using information received from the in-toilet device. Data acquired so far may be accumulated in the estimation means 200 or may be accumulated in the display means 300. The estimation means 200 generates information for estimating the health condition of the user (also referred to as “health estimation information”) or information related thereto based on an amount of the health-related gas and an amount of the odoriferous gas in the fecal gas of the user. The estimation means 200 calculates a score as the health estimation information of the user based on a ratio between the amount of the health-related gas and the amount of the odoriferous gas in the fecal gas of the user. For example, the estimation means 200 may use arbitrary information such as the ratio and a single odor. The above is merely an example, and the estimation means 200 may generate arbitrary information as the health estimation information of the user. For example, the estimation means 200 may generate the following information as the health estimation information of the user, or may generate the health estimation information based on the following processing result.

For example, the estimation means 200 may estimate information regarding a state of the intestine of the user from a measurement value. For example, the estimation means 200 may estimate information regarding a state of bacteria. In this case, for example, the estimation means 200 may estimate an occupancy rate of a certain bacterium, an amount and a ratio of good bacteria and bad bacteria, and the like. Furthermore, for example, the estimation means 200 may estimate a state of a metabolite. In this case, for example, the estimation means 200 may estimate amounts of a useful substance and a harmful substance, a ratio thereof, and the like. For example, the estimation means 200 may estimate a state of intestinal pH. In addition, the estimation means 200 may generate information obtained by scoring the above information or evaluating a quality. For example, the estimation means 200 may generate the above information as the health estimation information of the user.

Furthermore, for example, the estimation means 200 may generate information regarding the health condition of the user from the measurement value. In this case, for example, the estimation means 200 may generate a score for an intestinal environment of the user or evaluation information indicating a quality. For example, the estimation means 200 may generate information regarding the intestinal environment of the user. For example, the estimation means 200 may generate information regarding immunity of the user. For example, the estimation means 200 may generate information regarding leanness of the user. For example, the estimation means 200 may generate information regarding a cholesterol index. For example, the estimation means 200 may generate information regarding a metabolic score. For example, the estimation means 200 may generate the above information as the health estimation information of the user. Each example described above is merely an example, and the estimation means 200 is not limited to the above, and may generate various types of information related to the health condition of the user.

The estimation means 200 estimates that the user is healthier as the amount of the health-related gas in the fecal gas of the user is larger than the amount of the odoriferous gas based on the calculated ratio. The estimation means 200 estimates that the user is unhealthier as the amount of the odoriferous gas in the fecal gas of the user is larger than the amount of the health-related gas based on the calculated ratio. The above is merely an example, and the estimation means 200 may perform arbitrary estimation based on the calculated score. The estimation means 200 transmits information to be provided to the user to the display means 300. The estimation means 200 transmits the score calculated as the health estimation information of the user to the display means 300 used by the user.

The estimation means 200 is not limited to the cloud server (server device), and may be an arbitrary device. That is, any form of a device configuration and disposition of the estimation means 200 can be adopted as long as desired processing can be implemented. For example, the estimation means 200 may be a portable terminal (device) such as a notebook computer that can be carried by an administrator or the like of the biological information measurement system 1. Further, the estimation means 200 may be disposed in the toilet room R. For example, the estimation means 200 may be configured to be disposed in the toilet room R. For example, the toilet seat device 2 may have the function of the estimation means 200. In this case, the control device 100 may function as the estimation means 200.

The display means 300 is a display device (computer) that displays information to be provided to the user. For example, the display means 300 may be a user terminal (mobile terminal) owned by the user. In this case, the display means 300 is implemented by, for example, a smartphone, a mobile phone, a personal digital assistant (PDA), a tablet terminal, or a notebook personal computer (PC). For example, the display means 300 is communicably connected to a device included in the biological information measurement system 1 such as the estimation means 200 via a predetermined network in a wired or wireless manner.

The display means 300 transmits and receives information to and from the estimation means 200. The display means 300 receives information to be provided to the user from the estimation means 200. The display means 300 receives the score calculated as the health estimation information of the user from the estimation means 200. The display means 300 displays information including the score calculated as the health estimation information of the user.

In FIG. 3, the display means 300 displays the score calculated as the health estimation information of the user as an intestinal environment score of the user. For example, the display means 300 displays the intestinal environment score of the user in time series for each date and time of excretion. The display means 300 displays a target value of the score, information indicating a temporal change in the intestinal environment score of the user, and text information indicating the evaluation. For example, the display means 300 may request the estimation means 200 for information and display the information acquired from the estimation means 200.

The above is merely an example, and the biological information measurement system 1 can adopt any device configuration as long as desired processing can be implemented. In the biological information measurement system 1, the toilet seat device 2 may have a configuration other than the display means 300. For example, the toilet seat device 2 may include the measurement device 4, the control device 100, and the estimation means 200. Furthermore, for example, the display means 300 does not have to be included in the biological information measurement system 1, or may be included in the biological information measurement system 1. For example, in a case where the display means 300 is the operation device 30 of the toilet room R, the display means 300 may be included in the biological information measurement system 1. In this case, the operation device 30 has a function of displaying the health estimation information of the user.

### 1-4. Action of User and Operation of System

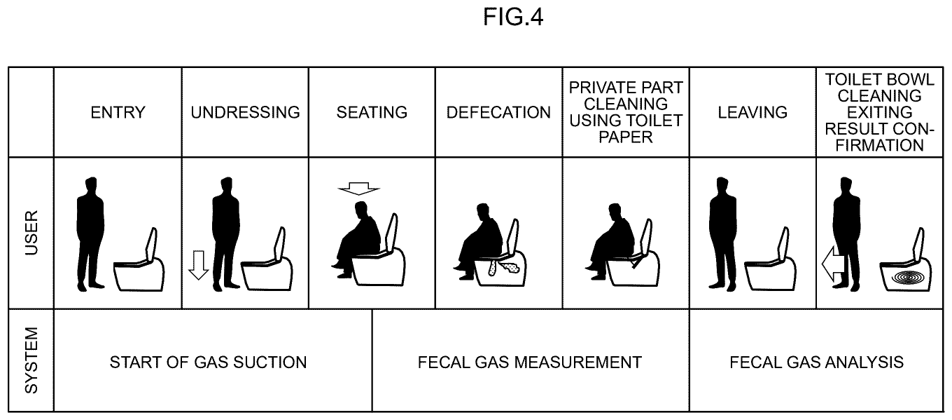

Next, an example of a relationship between a motion (action) of the user who uses the biological information measurement system 1 and a motion (operation) of the biological information measurement system 1 will be described with reference to FIG. 4. FIG. 4 is a diagram illustrating an example of the relationship between the action of the user and the operation of the system.

First, a flow of the action of the user who defecates using the toilet room R will be described with reference to FIG. 4. The user in the toilet room R performs actions of first to seventh stages as illustrated in FIG. 4.

First, the user performs an action of entering the toilet room R as the action of the first stage. The user who has entered the toilet room R performs an action of undressing in the toilet room R as the action of the second stage. The undressed user performs an action of being seated on the toilet seat 5 of the toilet room R as the action of the third stage. The user seated on the toilet seat 5 performs an action of defecating to the bowl portion 8 of the toilet bowl 7 as the action of the fourth stage.

The user who has defecated performs a finishing action such as cleansing the private part after defecation using the private part cleaning function of the toilet seat device 2 or toilet paper as the action of the fifth stage. The user who has completed the finishing after the defecation performs an action of rising and leaving the toilet seat 5 as the action of the sixth stage. The user who has left the seat cleans the toilet bowl 7, exits the toilet room R, confirm a fecal gas analysis result of the biological information measurement system 1, and the like as the action of the seventh stage.

Next, a flow of the operation of the biological information measurement system 1 corresponding to the action of the user described above will be described. The biological information measurement system 1 starts sucking the gas before the user who has entered the toilet room R starts to defecate. In FIG. 4, the biological information measurement system 1 starts the suction of the gas during a period between the first stage and the third stage. Therefore, the biological information measurement system 1 completes measurement preparation before the user defecates. For example, the biological information measurement system 1 sucks the gas mixture (gas) in the bowl portion 8 before the user defecates, thereby sucking a gas serving as a reference (baseline) for comparison with a gas after the defecation of the user. For example, the biological information measurement system 1 calculates an increment (increase amount) from the baseline and estimates (calculates) an amount of the component contained in the fecal gas.

The biological information measurement system 1 measures the fecal gas during a period in which the seated user defecates and leaves the seat. In FIG. 4, the biological information measurement system 1 measures the fecal gas of the user during a period from before the fourth stage to the fifth stage. As a result, the biological information measurement system 1 sucks the gas as needed while the user is seated and acquires data.

The biological information measurement system 1 analyzes the fecal gas after the measurement of the fecal gas is completed. In FIG. 4, the biological information measurement system 1 analyzes the fecal gas of the user during a period between the sixth stage and the seventh stage. As a result, after the user finishes the defecation, the biological information measurement system 1 performs the analysis and calculates the score based on information regarding the fecal gas (result) acquired for the user. The biological information measurement system 1 analyzes the fecal gas of the user and provides the analysis result to the user. The analysis and the provision of the result are not limited to being performed during a period from the sixth stage to the seventh stage, and may be performed at any timing as long as the information can be provided. For example, the biological information measurement system 1 may perform the analysis and provide various types of information such as the result at any timing such as during the measurement or immediately after completion of the measurement.

### 1-5. Functional Configuration of Toilet Seat Device

Next, a functional configuration of the toilet seat device 2 will be described with reference to FIG. 5. FIG. 5 is a block diagram illustrating an example of a configuration of the toilet seat device according to the embodiment. As illustrated in FIG. 5, the toilet seat device 2 includes a human sensor 32, a seating sensor 33, an illuminance sensor 34, the control device 100, a nozzle motor 61, and the cleaning nozzle 6.

The configuration of the toilet seat device 2 illustrated in FIG. 5 is merely an example, and the toilet seat device 2 may include only the toilet seat 5 in a case where each configuration is individually provided. As described above, the configuration of the toilet seat device 2 illustrated in FIG. 5 is merely an example, and any configuration can be adopted as the toilet seat device 2. The human sensor 32, the seating sensor 33, the illuminance sensor 34, and the like may be disposed at arbitrary positions as long as desired sensing is possible. Further, it is sufficient if the toilet seat device 2 can sense seating of the user on the toilet seat 5, and include at least one of the human sensor 32, the seating sensor 33, and the illuminance sensor 34. The toilet seat device 2 transmits and receives information to and from the information processing device such as the estimation means 200 in a wired or wireless manner via a predetermined network (the Internet or the like) by a communication device (for example, a communication unit 110 of the control device 100 in FIG. 6).

The human sensor 32 has a function of sensing a human body. For example, the human sensor 32 is used as seating sensing means that senses seating of the user on the toilet seat 5. For example, the human sensor 32 is implemented by a pyroelectric sensor using an infrared signal. For example, the human sensor 32 may be implemented by a microwave (μ) wave sensor or the like. For example, the human sensor 32 is an infrared projecting/receiving type distance measurement sensor, and may sense the human body present near the toilet seat 5 immediately before the person (user) is seated on the toilet seat 5 or the user seated on the toilet seat 5.

The human sensor 32 also functions as a seat leaving sensor that senses leaving of the user from the toilet seat 5. The human sensor 32 senses a seated state of the user on the toilet seat 5. The human sensor 32 outputs a sensing signal to the control device 100. The above is an example, and the human sensor 32 is not limited thereto and may sense the human body by various means. For example, the human sensor 32 senses a person (the user or the like) who approaches the toilet seat 5.

The seating sensor 33 has a function of sensing seating of a person on the toilet seat device 2. For example, the seating sensor 33 is used as the seating sensing means that senses seating of the user on the toilet seat 5. For example, the seating sensor 33 is implemented by a load sensor or the like. The seating sensor 33 senses that the user is seated on the toilet seat 5. The seating sensor 33 can sense seating of the user on the toilet seat 5.

The seating sensor 33 also functions as the seat leaving sensor that senses leaving of the user from the toilet seat 5. The seating sensor 33 senses the seated state of the user on the toilet seat 5. The above is an example, and the seating sensor 33 is not limited thereto and may sense seating of a person on the toilet seat device 2 by various means. The seating sensor 33 outputs a seating sensing signal to the control device 100.

The illuminance sensor 34 is a sensor that senses an illuminance. For example, the illuminance sensor 34 is used as the seating sensing means that senses seating of the user on the toilet seat 5. For example, the illuminance sensor 34 is disposed at a position facing the bowl portion 8, and senses the illuminance in the bowl portion 8.

The illuminance sensor 34 also functions as the seat leaving sensor that senses leaving of the user the toilet seat 5. The illuminance sensor 34 senses the seated state of the user on the toilet seat 5. The above is merely an example, and the illuminance sensor 34 may be disposed at any position as long as seating of the user on the toilet seat 5 can be sensed by the illuminance.

The control device 100 controls various configurations and processing. The control device 100 is a computer (information processing device) that executes various steps of information processing related to the measurement of the gas and the like. The control device 100 may be any device as long as the device has a configuration necessary for control, and may be, for example, a microcomputer.

The control device 100 controls various configurations for measuring the gas. The control device 100 controls the gas detection device 20. The control device 100 controls the gas detection device 20 such that the measurement value of the gas sensor 40 becomes a value within a predetermined range while the user does not use the toilet bowl 7, and performs reference value control for controlling the measurement value used as a reference value to a predetermined value. The control device 100 changes a resistance value of a resistor element of the gas sensor 40 to perform the reference value control for controlling the reference value to the predetermined value.

The control device 100 acquires defecation act use prediction information related to information for predicting a defecation act of the user, and performs the reference value control when the defecation act use prediction information is acquired. The control device 100 acquires the defecation act use prediction information based on the sensing by the seating sensing means, and performs the reference value control when the defecation act use prediction information is acquired.

The control device 100 transmits control information to the gas detection device 20 in a wired manner. The control device 100 may transmit the control information to the gas detection device 20 in a wireless manner. For example, in a case where the control device 100 is configured as a device separate from the toilet seat device 2, the control device 100 may wirelessly transmit the control information of the gas detection device 20 to the toilet seat device 2. In this case, the control device of the toilet seat device 2 may control the gas detection device 20 based on the received control information.

The control device 100 may control the suction device 10. For example, the control device 100 controls start and stop of the suction by the suction device 10. The control device 100 transmits control information to the suction device 10 in a wired manner. The control device 100 may transmit the control information to the suction device 10 in a wireless manner. For example, in a case where the control device 100 is configured as a device separate from the toilet seat device 2, the control device 100 may wirelessly transmit the control information of the suction device 10 to the toilet seat device 2. In this case, the control device of the toilet seat device 2 may control the suction device 10 based on the received control information.

In addition to the above, the control device 100 controls various configurations of the biological information measurement system 1. The control device 100 controls the nozzle motor 61 and the like. The control device 100 controls the nozzle motor 61 and the like based on a signal transmitted from the operation device 30.

The control device 100 controls the nozzle motor 61 based on a signal of a control instruction related to the private part cleaning transmitted from the operation device 30. The control device 100 controls the nozzle motor 61 to move the cleaning nozzle 6 forward and backward. The control device 100 may control various mechanisms in addition to the nozzle motor 61. For example, the control device 100 controls opening and closing of an electromagnetic valve having a function of a valve that controls a flow of a fluid by an electromagnetic method. For example, the control device 100 switches between supply and stop of tap water from a water supply pipe, for example, by controlling the electromagnetic valve.

The control device 100 transmits control information to the nozzle motor 61 and the like in a wired manner. The control device 100 may transmit the control information to the nozzle motor 61 and the like in a wireless manner. For example, in a case where the control device 100 is configured as a device separate from the toilet seat device 2, the control device 100 may wirelessly transmit the control information of the nozzle motor 61 and the like to the toilet seat device 2. In this case, the control device of the toilet seat device 2 may control the nozzle motor 61 and the like based on the received control information.

Further, the control device 100 may control the toilet lid 9 and the toilet seat 5 as illustrated in FIG. 1. In this case, the control device 100 controls the toilet lid 9 and the toilet seat 5 based on a signal transmitted from the operation device 30. The control device 100 controls the toilet lid 9 based on a signal of a control instruction related to opening and closing of the toilet lid transmitted from the operation device 30. The control device 100 controls the toilet seat 5 based on a signal of a control instruction related to opening and closing of the seating portion transmitted from the operation device 30. The control device 100 transmits control information to the toilet lid 9 and the toilet seat 5 in a wired manner. The control device 100 may transmit the control information to the toilet lid 9 and the toilet seat 5 in a wireless manner.

The control device 100 determines whether or not the seating of the user has been sensed by the seating sensing means such as the human sensor 32, the seating sensor 33, or the illuminance sensor 34. The control device 100 determines whether or not the seating of the user on the toilet seat 5 has been sensed based on the defecation act use prediction information based on the sensing by the seating sensing means acquired from the seating sensing means.

The nozzle motor 61 is the drive source (motor) that drives the cleaning nozzle 6 forward and backward. The nozzle motor 61 performs control to move the cleaning nozzle 6 forward and backward with respect to the main body portion 3. The nozzle motor 61 performs control to move the cleaning nozzle 6 forward and backward according to an instruction from the control device 100.

In the configuration illustrated in FIG. 5, a configuration in which the control device 100 and the like are included in the toilet seat device 2 is illustrated as an example. However, the control device 100, the human sensor 32, the seating sensor 33, the illuminance sensor 34, and the like may be configured as devices separate from the toilet seat device 2. For example, the control device 100 may be configured as a device separate from the toilet seat device 2. For example, the control device 100 may be a server device, and may be disposed at a position separated from the toilet seat device 2. In this case, the control device 100 communicates with each device such as the toilet seat device 2, the human sensor 32, the seating sensor 33, or the illuminance sensor 34, and receives various types of information from each device. In this case, the toilet seat device 2 may have a configuration (a control circuit or the like) for controlling various configurations of the toilet seat device 2 such as the nozzle motor 61. The above is merely an example, and the biological information measurement system 1 can adopt any device configuration as long as desired processing can be implemented.

### 1-6. Functional Configuration of Control Device

Hereinafter, a functional configuration of the control device will be described with reference to FIG. 6. FIG. 6 is a block diagram illustrating an example of a configuration of the control device according to the embodiment. As illustrated in FIG. 6, the control device 100 includes the communication unit 110, a storage unit 120, and a control unit 130. The configuration of the control device 100 is not limited to the configuration illustrated in FIG. 6, and may be another configuration as long as desired processing can be implemented. For example, the control device 100 does not have to include the communication unit 110.

The communication unit 110 is implemented by, for example, a communication circuit or the like. The communication unit 110 is connected to a predetermined network in a wired or wireless manner, and transmits and receives information to and from an external information processing device. For example, the communication unit 110 is connected to a predetermined network in a wired or wireless manner, and transmits and receives information to and from another device such as the operation device 30. The communication unit 110 may be configured as a device (communication device) separate from the control device 100, and may be included in the toilet seat device 2.

The storage unit 120 is implemented by, for example, a semiconductor memory element such as a random access memory (RAM) or a flash memory, or a storage device such as a hard disk or an optical disk. For example, the storage unit 120 is a computer-readable recording medium that non-temporarily records data and the like used by various information processing programs and the like.

The storage unit 120 according to the embodiment stores various types of information necessary for processing. The storage unit 120 stores various types of information acquired from other devices such as various sensors. The storage unit 120 stores various types of information used in various steps of information processing. For example, the storage unit 120 stores information regarding the reference value control such as the target value.

Returning to FIG. 6, the description will be continued. The control unit 130 is implemented by, for example, a micro processing unit (MPU), a central processing unit (CPU), or the like executing a program (for example, a program and the like for various steps of information processing according to the present disclosure) stored in the control device 100 using a RAM or the like as a work area. Furthermore, the control unit 130 may be implemented by, for example, an integrated circuit such as an application specific integrated circuit (ASIC) or a field programmable gate array (FPGA).

As illustrated in FIG. 6, the control unit 130 includes an acquisition unit 131, a processing unit 132, and an output unit 133, and implements or executes a function and an action of information processing described below. The internal configuration of the control unit 130 is not limited to the configuration illustrated in FIG. 6, and may be another configuration as long as information processing described below is executed.

The acquisition unit 131 acquires various types of information. The acquisition unit 131 acquires various types of information from the storage unit 120. The acquisition unit 131 receives information from another device. The acquisition unit 131 receives, from various sensors, information (sensing information and the like) sensed by various sensors.

The acquisition unit 131 acquires, from the seating sensing means, information (sensing information and the like) sensed by the seating sensing means. The acquisition unit 131 receives information (sensing information or the like) sensed by at least one of the human sensor 32, the seating sensor 33, and the illuminance sensor 34 from the sensor.

The acquisition unit 131 acquires the defecation act use prediction information based on the sensing by the seating sensing means. For example, the acquisition unit 131 acquires the defecation act use prediction information indicating the seating of the user.

The processing unit 132 performs various steps of processing. The processing unit 132 performs various steps of processing using the information stored in the storage unit 120. The processing unit 132 controls the gas detection device 20.

The processing unit 132 performs the reference value control for controlling the gas detection device 20 such that the measurement value of the gas sensor 40 becomes a value within the predetermined range while the user does not use the toilet bowl 7, and controlling the measurement value used as the reference value to be the predetermined value. The processing unit 132 performs the reference value control for controlling the reference value to be the predetermined value by changing the resistance value of the resistor element of the gas sensor 40. The processing unit 132 performs the reference value control every time the measurement of the fecal gas ends. The processing unit 132 performs the reference value control by processing of feeding back the measurement value of the gas sensor 40.

In a case where the acquisition unit 131 has acquired the defecation act use prediction information for predicting the defecation act of the user, the processing unit 132 performs the reference value control. In a case where the acquisition unit 131 has acquired the defecation act use prediction information indicating the seating of the user on the toilet seat 5, the processing unit 132 performs the reference value control.

The processing unit 132 executes determination processing. The processing unit 132 executes the determination processing by using various types of information stored in the storage unit 120. The processing unit 132 determines whether or not to perform the reference value control by using various types of information acquired by the acquisition unit 131.

The processing unit 132 executes calculation processing. The processing unit 132 executes the calculation processing by using various types of information stored in the storage unit 120. The processing unit 132 executes the calculation processing by using various types of information acquired by the acquisition unit 131.

The processing unit 132 calculates various types of information regarding the gas. The processing unit 132 calculates a value based on the measurement value measured by the gas detection device 20. The processing unit 132 calculates a resistance value of a sensor element based on a voltage value measured by the gas sensor 40. For example, the processing unit 132 calculates the resistance value of the sensor element from the measured voltage value by using a function indicating a relationship between the voltage value and the resistance value of the sensor element. The processing unit 132 calculates the resistance value of the sensor element using Expression (1).

The processing unit 132 may calculate a concentration of the gas based on the calculated resistance value of the sensor element. In this case, the processing unit 132 calculates the concentration of the gas from the calculated resistance value by using a function indicating a relationship between the resistance value and the concentration of the gas.

The output unit 133 performs output processing of outputting various types of information. The output unit 133 functions as a transmission unit that transmits various types of information. The output unit 133 performs the output processing by transmitting information to an external information processing device. The output unit 133 transmits information to the external information processing device. For example, the output unit 133 transmits various types of information to the estimation means 200. For example, the output unit 133 transmits various types of information to an administrator device such as a personal computer or a smartphone used by an administrator of the estimation means 200. Furthermore, the output unit 133 may perform the output processing by transmitting information to the operation device 30 (or the display screen 31).

The output unit 133 transmits various types of information used for the estimation processing by the estimation means 200 to the estimation means 200. The output unit 133 transmits information indicating the measurement value acquired by the gas detection device 20. The output unit 133 transmits information indicating the value calculated by the processing unit 132.

### 1-7. Gas Sensor

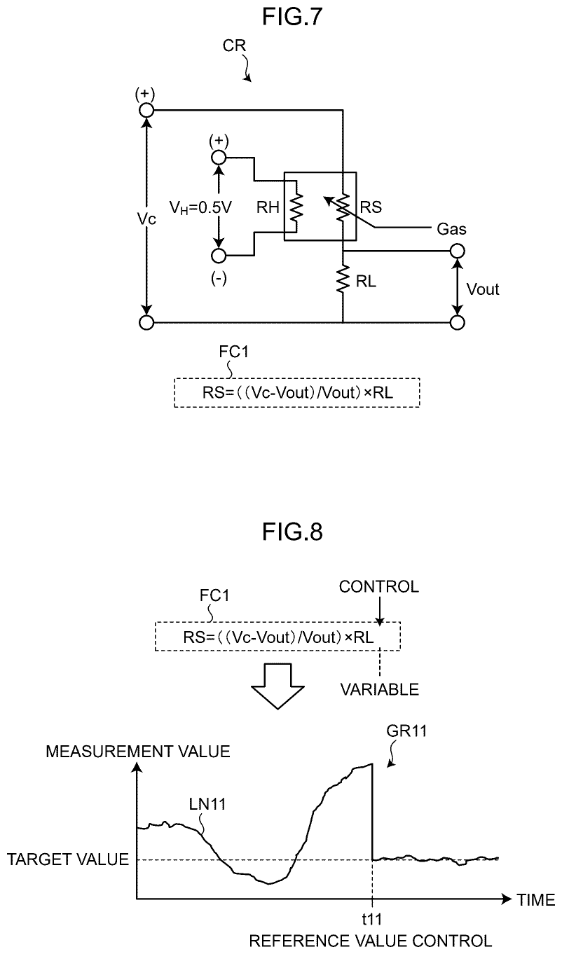

Hereinafter, a configuration example of the gas sensor will be described with reference to FIG. 7. FIG. 7 is a diagram illustrating an example of a configuration of the gas sensor. Specifically, FIG. 7 is a diagram illustrating an example of a circuit configuration CR of the semiconductor gas sensor 40.

The gas sensor 40 includes the sensor element and the resistor element for measurement. In FIG. 7, the gas sensor 40 has the circuit configuration CR in which the sensor element (corresponding to a sensor resistor RS in

FIG. 7) and the resistor element for measurement (corresponding to a resistor element RL in FIG. 7) are connected in series.

In the semiconductor gas sensor 40, a value related to a gas amount is calculated using the following Expression (1). Expression (1) corresponds to the circuit configuration CR illustrated in FIG. 7, and is an expression similar to a function FC1 in FIG. 7.

$$RS = ( ( Vc - V out ) / V out ) \times RL \qquad ( 1 )$$

“RS” in Expression (1) represents the resistance

value of the sensor element. For example, “RS” in Expression (1) indicates the resistance value of the sensor resistor RS, which is an example of a value calculated based on measurement by the gas sensor 40. As described above, Expression (1) is a calculation expression of the resistance value.

“RL” in Expression (1) represents the resistance value of the resistor element RL. “Vc” in Expression (1) represents a voltage value of a circuit voltage Vc. “Vout” in Expression (1) represents a voltage value of an output voltage Vout in the resistor element. For example, “Vout” in Expression (1) indicates a voltage value of the resistor element RL, which is an example of the measurement value acquired by the gas sensor 40.

The resistance value of the sensor resistor RS in Expression (1) is an index related to the gas amount. The biological information measurement system 1 calculates the index (resistance value) related to the gas amount from the measurement value (voltage value), and calculates the gas amount from the calculated resistance value. Although the principle and the like of the semiconductor gas sensor will not be described in detail, for example, “RH” illustrated only in the circuit configuration CR of FIG. 7 corresponds to a heater (resistor) for heating the sensor element, and “VH” corresponds to a voltage of the heater. The gas sensor in the present invention is not limited to a semiconductor sensor, and can be substituted by any sensor that satisfies Expression (1).

### 1-8. Reference Value Control

Hereinafter, a processing example related to the reference value control on the premise of the above-described configuration of the gas sensor 40 will be described. First, prior to the description of the processing related to the reference value control, a resolution change caused by a difference in reference value will be described. For example, the reference value is the measurement value (voltage value) before the measurement of the fecal gas, and is a value used as the baseline.

For example, an example in which the voltage value (measurement value) of the output voltage Vout changes from 2.5 V to 2.7 V will be described. In this example, for example, 2.5 V is the reference value (base line), and the voltage value is increased by 0.2 V from the reference value. First, in a case where the voltage value of the output voltage Vout is 2.5 V, the following Expression (2) is established.

$$RS = ( ( 5 - 2.5 ) / 2.5 ) \times RL \qquad ( 2 )$$

As illustrated in Expression (2), in a case where the voltage value of the output voltage Vout is 2.5 V, “RS=1RL”.

Next, in a case where the voltage value of the output voltage Vout is 2.7 V, the following Expression (3) is established.

$$RS = ( ( 5 - 2.7 ) / 2.7 ) \times RL \qquad ( 3 )$$

As illustrated in Expression (3), in a case where the voltage value of the output voltage Vout is 2.7 V, “RS=0.852RL”. As described above, in a case where the voltage value of the output voltage Vout changes from 2.5 V to 2.7 V, the resistance value of the sensor resistor RS decreases by about 14.8%.

For example, an example in which the voltage value (measurement value) of the output voltage Vout changes from 4.5 V to 4.7 V will be described. In this example, for example, 4.5 V is the reference value (base line), and the voltage value is increased by 0.2 V from the reference value. First, in a case where the voltage value of the output voltage Vout is 4.5 V, the following Expression (4) is established.

$$RS = ( ( 5 - 4.5 ) / 4.5 ) \times RL \qquad ( 4 )$$

As illustrated in Expression (4), in a case where the voltage value of the output voltage Vout is 4.5 V, “RS=0.11RL”.

Next, in a case where the voltage value of the output voltage Vout is 4.7 V, the following Expression (5) is established.

$$RS = ( ( 5 - 4.7 ) / 4.7 ) \times RL \qquad ( 5 )$$

As illustrated in Expression (5), in a case where the voltage value of the output voltage Vout is 4.7 V, “RS=0.06RL”. As described above, in a case where the voltage value of the output voltage Vout changes from 4.5 V to 4.7 V, the resistance value of the sensor resistor RS decreases by about 42.5%.

As described above, even with the same change of 0.2 V, the amount of change in the resistance value of the sensor resistor RS varies depending on the reference value. In this way, when the reference value changes, a resolution changes. Therefore, in order to suppress the change in the resolution and stabilize the resolution, it is desirable to suppress the change in the reference value.

On the other hand, the output of the reference value tends to change depending on a use environment of the gas sensor. A toilet space (for example, the space in the toilet room R) tends to have a lot of noise. Examples of the noise in the toilet space include an air freshener, a cleaning agent, an alcohol-based sanitizer, a perfume, the fecal gas from the previous user, a deodorizing fan flow rate, a temperature, a humidity, odors adhering to the toilet bowl, the motion of the user, opening and closing of the toilet lid, and opening and closing of a door. As the use environment of the gas sensor, air is continuously sent to the gas sensor by a suction device (for example, the suction device 10) at all times.

The measurement value (reference value) tends to change due to the two factors as described above. That is, the reference value and the resolution tend to change depending on the toilet space and the use environment of the gas sensor. When the resolution changes, an influence of the noise on the measurement changes, and it is thus difficult to appropriately execute the processing related to the gas measurement.

For example, in a case where the gas amount, the concentration, and the calculated score are evaluated by a temporal change, an influence of the resolution change on the evaluation is particularly large. For example, when the resolution changes for each measurement, the degree of influence of the noise changes, which affects the evaluation by the temporal change. That is, in a case where the evaluation is performed by the temporal change, there may be a case where the evaluation cannot be correctly performed if the resolution changes. For example, in a case where the gas amount, the concentration, and the calculated score are evaluated by the temporal change, the resolution change affects the evaluation.

Therefore, in order to stabilize the resolution, the biological information measurement system 1 performs the reference value control in order to suppress the change in the reference value. For example, the biological information measurement system 1 performs the reference value control for controlling the gas detection device 20 such that the measurement value of the gas sensor 40 becomes a value within the predetermined range while the user does not use the toilet bowl 7, and controlling the measurement value used as the reference value to be the predetermined value.

Then, the biological information measurement system 1 executes processing related to the fecal gas measurement by using the reference value controlled by the reference value control. For example, the gas detection device 20 executes processing related to the fecal gas measurement by using the reference value controlled by the reference value control performed by the control device 100.

### 1-8-1. Outline of Reference Value Control

Hereinafter, an example of the reference value control by the biological information measurement system 1 will be described. First, an outline of the reference value control will be described with reference to FIG. 8. FIG. 8 is a diagram illustrating an example of the reference value control according to the embodiment.

The biological information measurement system 1 changes the resistance value of the resistor element for measurement in order to suppress the change in the reference value. That is, in the circuit configuration CR of the semiconductor gas sensor 40, a variable resistor is used for the resistor element RL which is the resistor element for measurement. As illustrated in FIG. 8, the biological information measurement system 1 changes the resistance value of the resistor element RL such that the reference value (measurement value) becomes the predetermined value (target value). For example, the target value is set to a value that can be measured as the voltage value of the resistor element RL. The target value can be set to any value, and may be set to a value with the most favorable resolution.

A graph GR11 in FIG. 8 shows an example of control of the measurement value by the reference value control, in which a vertical axis represents the measurement value (the voltage value of the resistor element RL) and a horizontal axis represents the lapse of time. For example, a time point t11 of the graph GR11 corresponds to a time point before the start of the measurement of the fecal gas.

A line LN11 of the graph GR11 indicates the temporal change of the measurement value (the voltage value of the resistor element RL). As illustrated in FIG. 8, the biological information measurement system 1 performs the reference value control at the time point t11 before the start of the measurement of the fecal gas, and changes the resistance value of the resistor element RL such that the measurement value (the voltage value of the resistor element RL) serving as the reference value becomes the target value. The biological information measurement system 1 performs the reference value control at an arbitrary timing before the user starts the defecation. For example, the biological information measurement system 1 may sense the seating of the user by the seating sensing means or the like, and start the reference value control at a timing at which the seating of the user is sensed. The timing for the reference value control will be described below. Then, after the time point t11, the biological information measurement system 1 measures the fecal gas of the user based on the reference value (baseline) controlled by the reference value control.

As a result, the biological information measurement system 1 can suppress the change in the resolution by the reference value control even in a case where the measurement value changes. The biological information measurement system 1 can eliminate an influence of noise factors such as the temperature, the humidity, and the air freshener. The processing of the reference value control described above is merely an example, and the biological information measurement system 1 may perform the reference value control by an arbitrary mode at an arbitrary timing. For example, the biological information measurement system 1 may continuously perform the reference value control at a timing other than a timing at which the seating sensing means performs sensing. This point will be described below.

### 1-8-2. Example of Timing of Reference Value Control

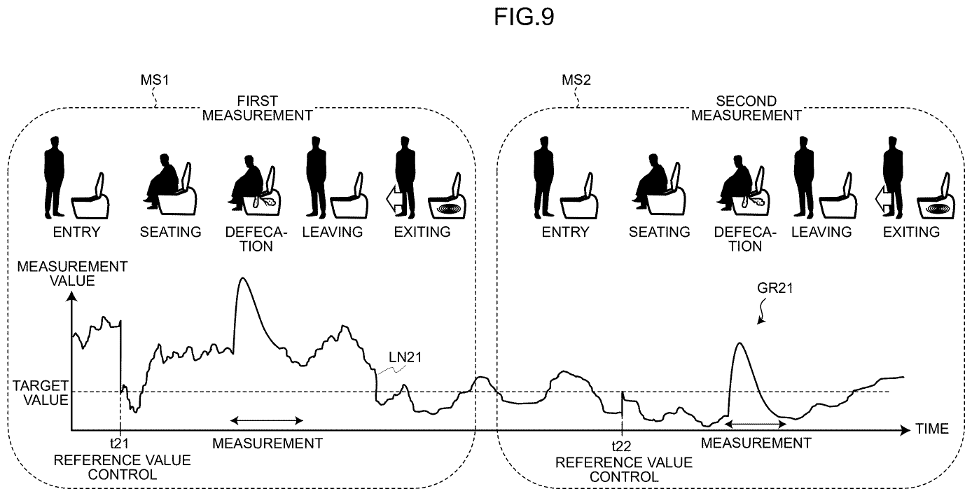

Next, an example of the timing of the reference value control will be described with reference to FIG. 9. FIG. 9 is a diagram illustrating an example of the timing of the reference value control according to the embodiment. A description of points similar to the contents described in FIG. 8 will be omitted as appropriate.

The biological information measurement system 1 performs the reference value control every time the measurement of the fecal gas ends. Specifically, the biological information measurement system 1 performs the reference value control every time before the measurement of the fecal gas.

A graph GR21 in FIG. 9 shows an example of the control of the measurement value by the reference value control for each fecal gas measurement performed by the biological information measurement system 1 according to the use of the toilet room R by the user. A line LN21 of the graph GR21 indicates the temporal change of the measurement value (the voltage value of the resistor element RL).

Measurement processing MS1 in FIG. 9 corresponds to the first measurement of the fecal gas among a plurality of times (two times) of fecal gas measurement illustrated in FIG. 9. For example, the measurement processing MS1 shows an example of the fecal gas measurement for the first user (also referred to as a “user U1”) among two times of uses of the toilet room R illustrated in FIG. 9. As illustrated in the measurement processing MS1, the user U1 uses the toilet room R by a procedure of entering the toilet room R, seating on the toilet seat 5, defecation to the bowl portion 8, leaving the toilet seat 5, and exiting the toilet room R.

For example, a time point t21 of the graph GR21 corresponds to a time point before the start of the measurement of the fecal gas of the user U1. As illustrated in FIG. 9, the biological information measurement system 1 performs the reference value control at the time point t21 before the measurement of the fecal gas of the user U1, that is, the first measurement of the fecal gas, and changes the resistance value of the resistor element RL such that the measurement value (the voltage value of the resistor element RL) serving as the reference value becomes the target value. For example, the biological information measurement system 1 may sense the seating of the user U1 by the seating sensing means or the like, and start the reference value control at a timing at which the seating of the user U1 is sensed. Then, after the time point t21, the biological information measurement system 1 measures the fecal gas of the user U1 based on the reference value (baseline) controlled by the reference value control.

Measurement processing MS2 in FIG. 9 corresponds to the second measurement of the fecal gas among the plurality of times (two times) of fecal gas measurement illustrated in FIG. 9. For example, the measurement processing MS2 shows an example of the fecal gas measurement for the second user (also referred to as a “user U2”) among two times of uses of the toilet room R illustrated in FIG. 9. As illustrated in the measurement processing MS2, the user U2 uses the toilet room R by a procedure of entering the toilet room R, seating on the toilet seat 5, defecation to the bowl portion 8, leaving the toilet seat 5, and exiting the toilet room R. The user U2 may be a user different from the user U1, or may be the same user as the user U1.

For example, a time point t22 of the graph GR21 corresponds to a time point before the measurement of the fecal gas of the user U2, that is, the start of the second fecal gas measurement. As illustrated in FIG. 9, the biological information measurement system 1 performs the reference value control at the time point t22 before the start of the measurement of the fecal gas of the user U2, and changes the resistance value of the resistor element RL such that the measurement value (the voltage value of the resistor element RL) serving as the reference value becomes the target value. For example, the biological information measurement system 1 may sense the seating of the user U2 by the seating sensing means or the like, and start the reference value control at a timing at which the seating of the user U2 is sensed. Then, after the time point t22, the biological information measurement system 1 measures the fecal gas of the user U2 based on the reference value (baseline) controlled by the reference value control.

As described above, the biological information measurement system 1 performs the reference value control every time before the measurement of the fecal gas. As described above, the biological information measurement system 1 can perform the reference value control every time until the next measurement of the fecal gas, and thus, can perform the measurement while suppressing the resolution change for every measurement of the fecal gas. The biological information measurement system 1 can eliminate the influence of the noise factors such as the fecal gas of the immediately previous user and the odors adhering to the toilet bowl.

### 1-8-3. Feedback Control of Reference Value

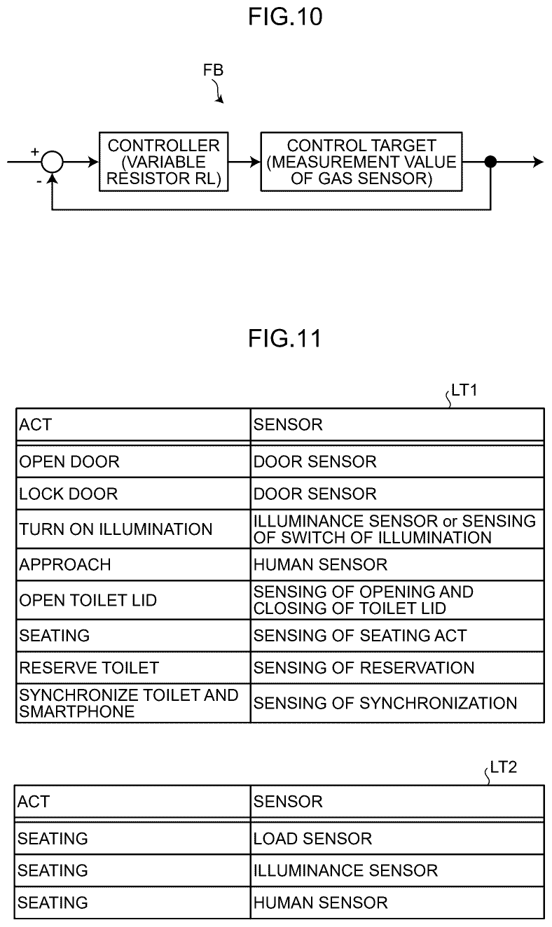

Next, feedback control of the reference value will be described with reference to FIG. 10. FIG. 10 is a diagram illustrating the feedback control of the reference value. The biological information measurement system 1 performs the reference value control by executing processing of feeding back the measurement value of the gas sensor 40.

As in feedback control FB illustrated in FIG. 10, the biological information measurement system 1 controls the variable resistor (resistor element RL) as a controller to control the measurement value of the gas sensor 40 to be controlled. As described above, the biological information measurement system 1 performs the reference value control on the resistor element RL that is the variable resistor by the feedback control using the measurement value of the gas sensor 40. Accordingly, the biological information measurement system 1 can perform control based on the current measurement value of the gas sensor 40.

### 1-8-4. Reference Value Control Before Defecation Act

Next, a specific example of the timing of the reference value control will be described with reference to FIG. 11. FIG. 11 is a diagram illustrating an example of actions before the defecation act. The biological information measurement system 1 senses a pre-defecation state based on a plurality of pre-defecation acts illustrated in a pre-defecation act list LT1 in FIG. 11 and performs the reference value control.

The biological information measurement system 1 may sense an act of opening the door of the toilet room R by the user as the pre-defecation act, and perform the reference value control for the toilet room R at a timing at which the act of opening the door of the toilet room R by the user is sensed. In this case, the biological information measurement system 1 includes a door sensor that senses the opening and closing of the door of the toilet room R, and acquires information indicating the act of opening the door of the toilet room R by the user based on the sensing performed by the door sensor.

The biological information measurement system 1 may sense an act of locking the door of the toilet room R by the user as the pre-defecation act, and perform the reference value control for the toilet room R at a timing at which the act of locking the door of the toilet room R by the user is sensed. In this case, the biological information measurement system 1 includes a door sensor that senses whether or not the door of the toilet room R is locked, and acquires information indicating the act of locking the door of the toilet room R by the user based on the sensing performed by the door sensor.

The biological information measurement system 1 may sense an act of turning on an illumination of the toilet room R by the user as the pre-defecation act, and perform the reference value control for the toilet room R at a timing at which the act of turning on the illumination of the toilet room R by the user is sensed. In this case, the biological information measurement system 1 may acquire information indicating the act of turning on the illumination of the toilet room R by the user based on the sensing performed by the illuminance sensor 34, or may acquire information indicating the act of turning on the illumination of the toilet room R based on an ON-OFF state of an illuminance switch.

The biological information measurement system 1 may sense an act of approaching the toilet seat 5 of the toilet room R by the user as the pre-defecation act, and perform the reference value control for the toilet room R at a timing at which the act of approaching the toilet seat 5 of the toilet room R by the user is sensed. In this case, the biological information measurement system 1 acquires information indicating the act of turning on the illumination of the toilet room R by the user based on the sensing performed by the human sensor 32.

The biological information measurement system 1 may sense an act of opening the toilet lid 9 of the toilet room R by the user as the pre-defecation act, and perform the reference value control for the toilet room R at a timing at which the act of opening the toilet lid 9 of the toilet room R by the user is sensed. In this case, the biological information measurement system 1 includes an opening/closing sensor that senses the opening/closing of the toilet lid 9 of the toilet room R, and acquires information indicating the act of opening the toilet lid 9 of the toilet room R by the user based on the sensing performed by the opening/closing sensor.

The biological information measurement system 1 may sense an act of being seated on the toilet seat 5 of the toilet room R by the user as the pre-defecation act, and perform the reference value control for the toilet room R at a timing at which the act of being seated on the toilet seat 5 of the toilet room R by the user is sensed.

In this case, the biological information measurement system 1 acquires information indicating the act of being seated on the toilet seat 5 of the toilet room R by the user based on the sensing performed by each sensor which is an example of the seating sensing means illustrated in a seating sensing list LT2 in FIG. 12.

In the seating sensing list LT2, a load sensor corresponds to the seating sensor 33, an illuminance sensor corresponds to the illuminance sensor 34, and a human sensor corresponds to the human sensor 32. The biological information measurement system 1 senses the seating of the user by the sensing performed by the seating sensing means, and starts the reference value control at a timing at which the seating of the user is sensed. In this way, by performing the reference value control at the timing of seating, the biological information measurement system 1 can perform the reference value control at the timing of seating at which a change in air quality is small before the defecation act, so that the reference value can be set more appropriately.

The biological information measurement system 1 may sense an act of reserving the toilet room R by the user as the pre-defecation act, and perform the reference value control for the toilet room R at a timing based on the act of reserving the toilet room R by the user. In this case, the biological information measurement system 1 acquires reservation information indicating the reservation of the toilet room R from an external device that manages the reservation of the toilet room R, and performs the reference value control for the toilet room R at a timing based on the acquired reservation information. For example, the biological information measurement system 1 may perform the reference value control for the toilet room R at a timing at which the reservation information is acquired. For example, in a case where a scheduled use start time is included in the reservation information, the biological information measurement system 1 performs the reference value control for the toilet room R immediately before (for example, several minutes before) the scheduled use start time.

The biological information measurement system 1 may sense an act of synchronizing a user terminal of the user with equipment (for example, the toilet seat device 2) in the toilet room R by the user as the pre-defecation act, and perform the reference value control for the toilet room R at a timing at which the act of synchronizing the user terminal with the equipment in the toilet room R by the user is sensed. In this case, the biological information measurement system 1 may perform the reference value control for the toilet room R at a timing at which synchronization start information indicating that the user terminal of the user and the equipment in the toilet room R start synchronization is acquired.

As described above, the biological information measurement system 1 performs the reference value control at a timing based on an arbitrary act among various acts as the pre-defecation act. As described above, since the biological information measurement system 1 can perform the reference value control before the defecation act, it is possible to perform the control more effectively. The biological information measurement system 1 can eliminate the influence of the noise factors such as the cleaning agent, the alcohol-based sanitizer, the opening and closing of the user, the motion of the user, the perfume, and the opening and closing of the toilet lid.

The above-described embodiment and modified examples can be appropriately combined within a range not resulting in contradictions in processing.

Further effects and modified examples can be easily derived by those skilled in the art. Therefore, broader aspects of the present invention are not limited to the specific details and representative embodiments presented and described above. Accordingly, various changes may be made without departing from the spirit or scope of the general inventive concept as defined by the appended claims and equivalents thereof.

The above-described embodiment and modified examples may have the following configurations, but are not limited to the following configurations.
(1)

A biological information measurement system that measures biological information of a user in a toilet room based on a fecal gas emitted into a bowl of a closet bowl installed in the toilet room, the biological information measurement system including:
a gas detection device that includes a gas sensor that reacts with a gas contained in a gas mixture; and
a control device that controls the gas detection device, in which
the gas sensor includes a sensor element and a resistor element for measurement,
the control device performs reference value control for controlling the gas detection device such that a measurement value of the gas sensor becomes a value within a predetermined range while the user does not use the closet bowl and controlling the measurement value used as a reference value to be a predetermined value, and
the gas detection device executes processing related to fecal gas measurement by using the reference value controlled by the reference value control.
(2)

The biological information measurement system according to (1), in which
the control device performs the reference value control for controlling the reference value to be the predetermined value by changing a resistance value of the resistor element of the gas sensor.

(3)

The biological information measuring system according to (1) or (2), in which
the control device performs the reference value control every time the fecal gas measurement ends.
(4)

The biological information measurement system according to (3), in which
the control device performs the reference value control by executing processing of feeding back the measurement value of the gas sensor.
(5)

The biological information measuring system according to (3) or (4), in which
the control device acquires defecation act use prediction information related to information for predicting a defecation act of the user, and performs the reference value control when the defecation act use prediction information is acquired.
(6)

The biological information measurement system according to (5), further including
seating sensing means that senses seating of the user on a toilet seat of the closet bowl, in which
the control device acquires the defecation act use prediction information based on the sensing performed by the seating sensing means, and performs the reference value control when the defecation act use prediction information is acquired.
(7)

A toilet seat device that measures biological information of a user in a toilet room based on a fecal gas emitted into a bowl of a closet bowl installed in the toilet room, the toilet seat device including:
a gas detection device that includes a gas sensor that reacts with a gas contained in a gas mixture; and
a control device that controls the gas detection device, in which
the gas sensor includes a sensor element and a resistor element for measurement,
the control device performs reference value control for controlling the gas detection device such that a measurement value of the gas sensor becomes a value within a predetermined range while the user does not use the closet bowl and controlling the measurement value used as a reference value to be a predetermined value, and
the gas detection device executes processing related to fecal gas measurement by using the reference value controlled by the reference value control.

### REFERENCE SIGNS LIST

1 BIOLOGICAL INFORMATION MEASUREMENT SYSTEM
2 TOILET SEAT DEVICE
3 MAIN BODY PORTION
4 MEASUREMENT DEVICE
5 TOILET SEAT
6 CLEANING NOZZLE
7 TOILET BOWL
8 BOWL PORTION
9 TOILET LID
10 SUCTION DEVICE
20 GAS DETECTION DEVICE
40 GAS SENSOR
100 CONTROL DEVICE
110 COMMUNICATION UNIT
120 STORAGE UNIT
130 CONTROL UNIT
131 ACQUISITION UNIT
132 PROCESSING UNIT
133 OUTPUT UNIT
200 ESTIMATION MEANS
R TOILET ROOM

What is claim is:

1. A biological information measurement system that measures biological information of a user in a toilet room based on an intestinal gas emitted into a bowl of a closet bowl installed in the toilet room, the biological information measurement system comprising:
a gas detection device that includes a gas sensor that reacts with a gas contained in a gas mixture; and
a control device that controls the gas detection device, wherein
the gas sensor includes a sensor element and a resistor element for measurement,
the control device performs reference value control for controlling the gas detection device such that a measurement value of the gas sensor becomes a value within a predetermined range while the user does not use the closet bowl and controlling the measurement value used as a reference value to be a predetermined value, and
the gas detection device executes processing related to intestinal gas measurement by using the reference value controlled by the reference value control.

2. The biological information measurement system according to claim 1, wherein
the control device performs the reference value control for controlling the reference value to be the predetermined value by changing a resistance value of the resistor element of the gas sensor.

3. The biological information measurement system according to claim 1, wherein
the control device performs the reference value control every time the intestinal gas measurement ends.

4. The biological information measurement system according to claim 3, wherein
the control device performs the reference value control by executing processing of feeding back the measurement value of the gas sensor.

5. The biological information measurement system according to claim 3, wherein
the control device acquires defecation act use prediction information related to information for predicting a defecation act of the user, and performs the reference value control when the defecation act use prediction information is acquired.

6. The biological information measurement system according to claim 5, further comprising
seating sensing means that senses seating of the user on a toilet seat of the closet bowl, wherein
the control device acquires the defecation act use prediction information based on the sensing performed by the seating sensing means, and performs the reference value control when the defecation act use prediction information is acquired.

7. A toilet seat device that measures biological information of a user in a toilet room based on an intestinal gas emitted into a bowl of a closet bowl installed in the toilet room, the toilet seat device comprising:
a gas detection device that includes a gas sensor that reacts with a gas contained in a gas mixture; and
a control device that controls the gas detection device, wherein
the gas sensor includes a sensor element and a resistor element for measurement,
the control device performs reference value control for controlling the gas detection device such that a measurement value of the gas sensor becomes a value within a predetermined range while the user does not use the closet bowl and controlling the measurement value used as a reference value to be a predetermined value, and
the gas detection device executes processing related to intestinal gas measurement by using the reference value controlled by the reference value control.
